(19) United States
(12) Patent Application Publication
(10) Pub. No.: US 2025/0258185 A1
(43) Pub. Date: Aug. 14, 2025

(54) METHODS AND APPARATUS FOR MEASURING INSULIN RESISTANCE BY DETECTION OF NORMAL AND ABNORMAL INSULIN ISOMERS AND MODULATION OF INSULIN RESISTANCE TREATMENT

(71) Applicants: Andreea Oana Mocanu, Easton, CT (US); Andrei Mircea Mocanu, Easton, CT (US)

(72) Inventors: Andreea Oana Mocanu, Easton, CT (US); Andrei Mircea Mocanu, Easton, CT (US)

(21) Appl. No.: 18/739,035

(22) Filed: Jun. 10, 2024

Related U.S. Application Data
(60) Provisional application No. 63/569,993, filed on Mar. 26, 2024.

(51) Int. Cl. G01N 33/74

(52) U.S. Cl. CPC G01N 33/743; G01N 2333/62; G01N 2440/20; G01N 2800/042

(57) ABSTRACT

The present invention is generally directed to methods for measuring Insulin Resistance (IR) and for modulation of Type 2 Diabetes (T2D) onset treatment. In particular, diagnosis of IR is based on detection of different forms of disulfide bonds pairs that may form in insulin, affecting the normal insulin structure. Abnormal insulin is defined as any insulin that contains at least one of the forms of “abnormal” disulfide bond pairs. The methods herein measure human insulin isomers' concentration in a sample by utilizing HPLC or a monoclonal/polyclonal antibody to human insulin that contains “normal” or one or more “abnormal” disulfide bonds combinations. Some aspects of the invention also provide kits adapted for use in such methods. The human insulin isomers' concentration results are fed into a system to regulate the level of exogenous insulin needed to augment the level of normal insulin detected by the methods for a specific patient, or effectively regulate the amount of metformin, sulfonylureas, thiazolidinediones (PPARy agonists; glitazones), GLP-1s, SGLT-2 inhibitors and DPP-4 inhibitors or other IR treatment to be administered to the patient. The system and methods comprise a feedback loop to maintain a therapeutic level of insulin, metformin or other IR treatment and decrease the risk of hypoglycemia and other complications. The current invention has the advantage of being a self-referencing IR assessment by utilizing the measurements of “normal” insulin compared to “abnormal” insulin and not relying only on glucose measurements, Body Mass Index or other references included in current IR measurement methods. This leads to a much more responsive and faster treatment for IR compared to current approaches, which employ methods such as, Oral Glucose Tolerance Test (OGTT), which takes hours to perform.

### BACKGROUND OF THE INVENTION

The number of patients suffering from T2D continues to increase at a very high rate, becoming one of the most serious worldwide social problems (Int. Diabetes Fed., Diabetes Atlas, 2015). IR is one of the most important, serious factors prior to developing T2D. Pathogenesis of IR means insufficiency of insulin action on glucose uptake in skeletal muscle (Abdul-Ghani et al, “Pathogenesis of IR in skeletal muscle.”, J Biomed Biotech, 476279, 2010). IR mainly presents as a hyperinsulinemia.

The cause of IR is theoretically because of secretion of abnormal forms of insulin, or cellular unresponsiveness to the action of insulin (Reaven et al, “The Role of IR in the Pathogenesis of Diabetes Mellitus. Advances in metabolic disorders”, Academic Press. 9: 313-331. 1978). The clinical investigation of different stages of IR requires expertise in sophisticated “in vivo” and “in vitro” methods. At present the hyperinsulinemic euglycemic clamp is the reference method for quantifying insulin resistance. It is the gold standard for the measurement of insulin sensitivity.

Insulin sensitivity may be estimated using other methods—OGTT, HOMA-IR, GUIKI INDEX etc.—but all of these are either time consuming, especially for patients, or difficult for clinicians to select the most appropriate index for their studies/investigations (Gutch et al, “Assessment of insulin sensitivity/resistance.”, Indian J Endocrinol Metab 19(1): 160-164, 2015).

Insulin is the hormone secreted by the endocrine pancreas and is essential for regulating glucose metabolism in the body. Failure of insulin control results in Diabetes Mellitus (DM). Insulin represents the only medicine that treats DM. Recently, Diabetes Mellitus was defined as a “cluster of 5 diseases”, among which Insulin Resistance is one of them. One of these 5 stages of T2D refers to severe insulin-resistant diabetes patients, whose pancreas produces insulin, but the body does not respond to this insulin. These patients are insulin-resistant and present hyperinsulinemia.

Insulin resistance, currently, is defined as a “predictor” for the development of Type 2 Diabetes and is considered the “pre-disease stage” of Type 2 Diabetes. Many methods and indices are available for measuring IR (IR), but only few are used in clinical practice (Home-IR, QUIKI, and Matsuda), while the other methods are used for epidemiological/research purposes (Gutch et al, “Assessment of insulin sensitivity/resistance.”, Indian J Endocrinol Metab 19(1): 160-164, 2015)

Some clinicians use different indices of insulin sensitivity, based on the interrelations between concentration of insulin, glucose and other parameters obtained during an Oral Glucose Tolerance Test (OGTT). However, in clinical practice, their application is limited, due to the lack of exact reference values for OGTT and the time it takes to run this test. Therefore, due to drawbacks of current methods, and lack of new methods, the aim of the present invention is to introduce a new method of measuring insulin resistance.

Insulin is a small, globular peptide hormone, composed of 51 amino acids (human insulin protein) and has 2 chains, designated A-chain (21 residues) and B-chain (30 residues). The A-chain and B-chain are linked together by disulfide bonds. Usually, disulfide bonds form between —SH (thiol groups) of 2 Cysteine (Cys) amino acids. Disulfide bond formation is one of the most common crosslinks between peptide chains.

In proteins, disulfide bonds, or disulfide bridges (—S—S—) are covalent bonds that form between 2 thiol groups of Cys residues, through the oxidative folding process. Disulfide bonds are responsible for the structure, stability and function of the proteins in which they form.

Disulfide bonds may break and reform with incorrect pairings, resulting in an alteration of the 3-dimensional structure of a protein, followed by corresponding change in biological activity (Bhatia, “Chapter 8.”, Academic Press: 293-331. 2015). A peptide chain that has more than one disulfide bond can enter into “disulfide exchange” reactions, leading to shuffling/scrambling of disulfide bridge and thereby can change (affect) protein conformation and possibly function. These re-formed disulfide bonds may be just a temporary event. Also, they can be intermolecular (between monomers), with critical consequences for the conformational transition of the insulin molecule. Protein folding occurs through a fast reaction in which transient intermediaries in the insulin molecule's folding “journey” are very dynamic, short lived and very difficult to isolate, or even trap.

Folding pathways of many proteins exhibit Bovine Pancreatic Trypsin Inhibitor-like (BPTI) and Hirudin-like mechanisms. One of these scenarios could be the case for insulin. The monomer of insulin has 3 (—S—S—), one is intra A-chain (A6-A11), and 2 inter B-chain (A7-B7 and A20-B19), which will “glue” the 2 chains together.

As a rule, any two Cysteine residues in a protein have the potential to form a disulfide bond. With two Cysteines in a protein, only one intramolecular disulfide bond can be formed; with three cysteines, three different disulfides could be formed; with four cysteines, six different disulfides, etc. Because a protein could have none, some, or all of its Cysteines in disulfide bonds, this gives 10 different intramolecular thiol/disulfide redox states for a protein with just 4 Cysteine residues (Hatahet et al, “Protein disulfide isomerase.”, Antioxid Redox Signal 11(11): 2807-2850. 2009). Usually, a protein will have only one pattern of disulfide bonds and free thiols in the normal state (unless it is involved in redox or it has redox-regulated activity) (Hatahet et al, “Protein disulfide isomerase.”, Antioxid Redox Signal 11(11): 2807-2850. 2009). A protein can attain its “normal disulfide state” through a folding pathway, where predominantly normal disulfide bonds are formed in the folding protein residues (Weissman and Kim 1991, Hatahet et al, “Protein disulfide isomerase.”, Antioxid Redox Signal 11(11): 2807-2850. 2009) and corresponds to the formation of regular secondary structure. Normally, the formation of the secondary structure will limit the juxtaposition of Cytosine residues in the folding intermediates to normal-like combinations. However, many proteins presumably form both normal and abnormal disulfide bonds during oxidation, and further processes are necessary to convert the abnormal disulfide bonds into normal ones. These were described to occur via two distinct pathways: 1) cycles of reduction-oxidation, and 2) direct isomerization. PDI catalyzes both pathways (Hatahet et al, “Protein disulfide isomerase.”, Antioxid Redox Signal 11(11): 2807-2850. 2009).

The kinetics of non-catalyzed folding pathways of disulfide bond containing proteins are probably determined by a combination of the pKa values (defined as the pH at which 50% of the species in protonated state, and 50% are in the deprotonated state) of individual Cysteines in the folding proteins, along with accessibility and the constraints put on the juxtaposition of pairs of Cysteine residues by the formation of regular secondary structural elements. The pKa values of the Cysteine residues will depend on the conformation of the folding intermediates, which cannot be easily verified (Hatahet et al, “Protein disulfide isomerase.”, Antioxid Redox Signal 11(11): 2807-2850. 2009). However, it is notable that the abnormal disulfide bonds observed by Creighton in the refolding pathway of BPTI (Creighton, “Protein folding pathways determined using disulphide bonds.”, Bioessays 14(3): 195-199, 1992), all contain Cysteine residues with adjacent positively charged residues, suggesting that a low pKa value for these cysteines may be responsible for the rapid intramolecular rearrangements that occur during quenching (Creighton, “Protein folding pathways determined using disulphide bonds.”, Bioessays 14(3): 195-199, 1992). So far, it is known that, in the relative reduction potential, defined as [H⁺]=10⁻⁷M, or pH=7.0 is defined relative Glutathione. PDI is defined as a protein oxidant (reduction potential of −180 mV) and will be preferentially reduced. It is very important to note that the reduction potential of a species cannot be seen in isolation, or only being reduced to the reaction between two species, ignoring the contributions from other species present in the respective environment. In vivo, the reduction potential of the cytoplasm for thiol-disulfide exchange reactions is defined by the major redox species present (reduced glutathione) (Hatahet et al, “Protein disulfide isomerase.”, Antioxid Redox Signal 11(11): 2807-2850. 2009).

Similarly, the reduction potential of the endoplasmic reticulum (ER) is defined primarily by the ratio of the major redox species present, reduced Glutathione (GSH) and oxidized Glutathione disulfide (GSSG), although all redox species present in the ER contribute. However, all the thioredoxin-superfamily members, including PDI, have the potential to act as catalysts of PDI bond formation, reduction, and isomerization, being dependent on the reduction potential of their active sites, on their kinetic properties, and on their physiological redox environment (Hatahet et al, “Protein disulfide isomerase.”, Antioxid Redox Signal 11(11): 2807-2850. 2009). Any conformational change in PDI, or due to substrate binding, or due to changes in the biophysical conditions can result in changing the reduction potential of the other sites.

In recent years, it has been found that even for small two-state proteins (˜100 amino acids or less), there exist partially unfolded intermediaries on the folding pathways (Zhou et al., “Detection of a hidden folding intermediate in the focal adhesion target domain: Implications for its function and folding.”, Proteins 65(2): 259-265, 2006; Kato et al., “The folding pathway of T4 lysozyme: the high-resolution structure and folding of a hidden intermediate.”, J Mol Biol 365(3): 870-880, 2007). However, growing evidence has indicated that the intermediate states formed during protein folding and unfolding may have significant roles in protein function by exposing post-translational modifications or ligand binding sites.

Disulfide bonds are formed between the sulfhydryl (—SH) side chains of two Cysteine residue: an S-anion from one (—SH) group acts as a nucleophile, attacking the side chain of a second Cys to create a disulfide bond (—S—S—). Proper disulfide bonds provide stability to a protein, decreasing further entropic choices that facilitates folding progression toward the normal state by limiting unfolded, or improperly folded conformations (Rajpal et al., “Disulfide Bond Formation. Handbook of Biologically Active Peptides”, Academic Press, 2013). The chemistry of protein (insulin) disulfide bond formation is directly influenced by three key factors:
the spatial accessibility/physical proximity of the partner Cysteine residues forming the disulfide bond,
the difference between the pKa of the involved thiol groups and the pH of the local environment (with lower pH limiting reactivity and higher pH favoring increased reactivity), and
the redox environment (with lesser reactivity under more reducing conditions and greater reactivity under more oxidizing conditions) (Mamathambika et al, “Disulfide-linked protein folding pathways.”, Annu Rev Cell Dev Biol 24: 211-235, 2008)

The last two factors are environmentally controlled, and specific cellular components have evolved, in order to accommodate the process of disulfide bond formation. In eukaryotes, the process occurs, mainly, in the endoplasmic reticulum (ER), although disulfide bond formation can, occasionally occur at other sites (including extracellularly). The ER is a vastly more common site than the cytosol (which is very rarely the site of disulfide bond formation), because the ER intraluminal environment is more oxidizing than that of the cytosol (Appenzeller-Herzog, “Glutathione—and non-glutathione—based oxidant control in the endoplasmic reticulum”, J Cell Sci 124(Pt 6): 847-855, 2011). The redox environment is, at least, partly reflected by the ratio of oxidized glutathione (GSSG) to reduced glutathione (GSH), which in the case of the ER, and is shifted in favor of GSSG.

Thiol-disulfide exchange results into formation of an intermediate in which the charge is shared among the three sulfur atoms. The thiolate group attacks a sulfur atom of the disulfide bond, displacing the other sulfur atom and forming a new disulfide bond (Bhatia, “Chapter 8.”, Academic Press: 293-331. 2015).

Disulfide bonds may break and reform with incorrect pairings. This results in an alteration in the three-dimensional structure followed by a change in biological activity. A peptide chain (in this hypothesis, pro-insulin), with more than one bisulfide bond, can enter into disulfide exchange reactions, leading to scrambling (isomerization) of disulfide bridges, and thereby, a change in conformation and function.

External perturbations in the form of pH change, thermal, or oxidative/reducing stress can destabilize the protein (insulin), resulting in misfolding and aggregation. Prolonged environmental stress can affect the cells adaptive response, resulting in loss of ability to refold or recycle proteins. This can lead to accumulation of misfolded or aggregated proteins (different forms of “new” insulins) within the cell/pancreas/blood (Dutta, “Effect of disulfide bond scrambling on protein stability, aggregation and cytotoxicity. Chapter 4”, Dept. of Chemistry, Michigan Technological University, 2016).

Disulfide bonds can do more than stabilize a normal structure; they can hold together a normal structure that is “frustrated” (Zhang, et al, “Disulfide Bridges: Bringing Together Frustrated Structure in a Bioactive Peptide.”, Biophys J 110(8): 1744-1752, 2016). Disulfide bonds usually cross-link distant regions of the peptide sequence, decreasing the entropy of the unfolded state and making it less favorable relative to the unfolded conformation (Wedemeyer et al, “Conformational propensities of protein folding intermediates . . . ”, Biochem 41(5): 1483-1491, 2002; Arolas et al., “Designing out disulfide bonds of leech carboxypeptidase inhibitor: implications for its folding, stability and function”, J Mol Biol 392(2): 529-546, 2009). Insulin structure changes with change in pH (Dutta, “Effect of disulfide bond scrambling on protein stability, aggregation and cytotoxicity. Chapter 4”, Dept. of Chemistry, Michigan Technological University, 2016)., with disulfide bonds scrambling at acidic pH.

In a protein, any two Cysteine residues have the potential to form a disulfide bond, only one intramolecular disulfide; three Cysteine residue will result in three different disulfide bonds formation; four Cysteine, results in six different disulfides; five Cysteine will result in eight disulfide bonds formation, etc. (Hatahet et al, “Protein disulfide isomerase.”, Antioxid Redox Signal 11(11): 2807-2850. 2009). Insulin has six Cysteine residues, which means there is a possibility of ten different disulfide bonds formation.

Because a protein could have none, some, or all of its Cysteine in disulfide bonds, this gives ten different intramolecular thiol/disulfide redox states for a protein with just four Cysteine residues and >13,000 different intramolecular redox states for a protein with ten Cysteine residues (Hatahet et al, “Protein disulfide isomerase.”, Antioxid Redox Signal 11(11): 2807-2850. 2009). If a protein cannot attain its normal disulfide state, meaning that “perturbations” can affect the folding pathway of the normal disulfides, it results in limitations of regular secondary structure formation. The result will be the development of intermediates to normal-like combinations. Or, many proteins, presumably, from both normal and nonnormal disulfide bonds during oxidation, and processes such as cycles of oxidation/reduction and direct isomerization, are necessary to convert the nonnormal disulfide bonds into normal ones. Both processes are catalyzed by PDI.

There are 15 possible disulfide bonds that can potentially form in insulin, as follows:
Intrachain A
   Cys (6)-Cys (11)
   Cys (6)-Cys (7)
   Cys (7)-Cys (11)
   Cys (6)-Cys (20)
   Cys (7)-Cys (20)
   Cys (11)-Cys (20)
Interchain A and B
   Cys (6)-Cys (7)
   Cys (7)-Cys (7)
   Cys (11)-Cys (7)
   Cys (20)-Cys (7)
   Cys (6)-Cys (19)
   Cys (7)-Cys (19)
   Cys (11)-Cys (19)
   Cys (20)-Cys (19)
Intrachain B
   Cys (7)-Cys (19)

Studies of a single-chain proinsulin analogue suggest that disulfide pairing occurs via a preferred kinetic pathway (Qiao et al, “Putative disulfide-forming pathway of porcine insulin precursor during its refolding in vitro.”, Biochemistry 40(9): 2662-2668, 2001, Hua et al, “A protein caught in a kinetic trap: structures and stabilities of insulin disulfide isomers.”, Biochem 41(50): 14700-14715, 2002).

The concentration of H2S in plasma or in tissue is regulated at the level of its generation and its consumption, and the levels are maintained within a certain range. Significant changes in the levels contribute to various diseases (Liu et al, “Disulfide bond structures of IgG molecules”, MAbs 4(1): 17-23, 2012).

According to Patel & Shah in their study (Patel et al, “Possible role of hydrogen sulfide in insulin secretion and in development of insulin resistance.”, J Young Pharm 2(2): 148-151, 2010) after administration of H2S in Wistar rats, the blood glucose level increased and insulin level decreased in the first 5 hours post H2S. In ZDF model, H2S concentration was reported to be higher than in Zucker lean rats (Jia et al, “The role of hydrogen sulfide in insulin resistance”, Canadian cardiovascular congress, Toronto, 2004). Also, expressions of both enzymes responsible for H2S production—CSE and CBS—were high in streptozotocin-induced diabetic rats and elevated in patients with T2D (Marathe et al, “A split dose regimen of streptozotocin to induce diabetes in a neonatal rat model”, Indian J Pharmacol., (38): 432-433, 2006). An important observation was that chronic treatment of H2S for 1 month had no significant effects on insulin sensitivity and that the high H2S level in diabetes is due to over-expression of H2S (Patel et al, “Possible role of hydrogen sulfide in insulin secretion and in development of insulin resistance.”, J Young Pharm 2(2): 148-151, 2010).

### FIELD OF THE INVENTION

The present invention is generally directed to methods for measuring Insulin Resistance by utilizing monoclonal/polyclonal antibody or HPLC to human insulin and kits adapted for use in such methods for modulation of Type 2 Diabetes onset treatment.

### SUMMARY OF THE INVENTION

The present disclosure is directed to methods for measuring insulin resistance and treatment of this disease. In particular, diagnosis of insulin resistance is based on detection of different forms of disulfide bonds pairs that may form in insulin, affecting the normal insulin structure. Abnormal insulin is defined as any insulin that contains at least one of the forms of “abnormal” disulfide bond pairs. The methods measure human insulin isomers' concentration in a sample, by utilizing HPLC or a monoclonal/polyclonal antibody to human insulin that contains “normal” or one, or more “abnormal” disulfide bonds combinations. The human insulin isomers' concentration results are fed into a system to regulate the level of exogenous insulin needed to augment the level of normal insulin detected by the methods for a specific patient, or effectively regulate the amount of Metformin, sulfonylureas, thiazolidinediones (PPARy agonists; glitazones), GLP-1s, SGLT-2 inhibitors and DPP-4 inhibitors or other Insulin Resistance treatment to be administered to the patient. The system and methods comprise of a feedback loop to maintain a therapeutic level of insulin, Metformin or other IR treatment and decrease the risk of hypoglycemia and other complications. The current disclosure has the advantage of being a self-referencing insulin resistance assessment by utilizing a concentration of “normal” insulin as compared to “abnormal” insulin and not needing glucose concentrations, Body Mass Index or other references included in current insulin resistance measurement methods. This leads to a much more responsive and faster treatment for insulin resistance compared to current approaches, which employ methods, such as Oral Glucose Tolerance Test (OGTT), which takes hours to perform.

### BRIEF DESCRIPTION OF THE DRAWINGS

FIG. 1 shows Disulfide Bond Structures in normal insulin. FIGS. 2-7 show abnormal Disulfide Bond Structures in normal insulin. FIG. 2 shows abnormal Disulfide Bond Structures in insulin isoform NCI1. FIG. 3 shows abnormal Disulfide Bond structures in insulin isoform NCI2. FIG. 4 shows abnormal Disulfide Bond Structures in insulin isoform NCI3. FIG. 5 shows abnormal Disulfide Bond Structures in insulin isoform NCI4. FIG. 6 shows abnormal Disulfide Bond Structures in insulin isoform NCI5. FIG. 7 shows abnormal Disulfide Bond Structures in insulin isoform NCI6.

FIG. 1 is a schematic representation of the sequence of human normal insulin CI01 isoform including the A-and B-chains showing the connecting Disulfide Bond Structures with intrachain A Cys(6)-A Cys(11) linkage, interchain A Cys(7)-B Cys(7) linkage, and interchain A Cys(20)-B Cys(19) linkage.

FIG. 2 is a schematic representation of the sequence of the abnormal Disulfide Bond Structures of abnormal human insulin NCI1 isoform including the A-and B-chains showing the connecting Disulfide Bond Structures with intrachain A Cys(6)-A Cys(7) linkage, interchain A Cys(11)-B Cys(7) linkage, and interchain A Cys(20)-B Cys(19) linkage.

FIG. 3 is a schematic representation of the sequence of the abnormal Disulfide Bond Structures of abnormal human insulin NCI2 isoform including the A-and B-chains showing the connecting Disulfide Bond Structures with interchain A Cys(6)-B Cys(7) linkage, intrachain A Cys(7)-A Cys(11) linkage, and interchain A Cys(20)-B Cys(19) linkage.

FIG. 4 is a schematic representation of the sequence of the abnormal Disulfide Bond Structures of abnormal human insulin NCI3 isoform including the A-and B-chains showing the connecting Disulfide Bond Structures with intrachain A Cys(6)-A Cys(20) linkage, interchain A Cys(7)-B Cys(7) linkage, and interchain A Cys(11)-B Cys(19) linkage.

FIG. 5 is a schematic representation of the sequence of the abnormal Disulfide Bond Structures of abnormal human insulin NCI4 isoform including the A-and B-chains showing the connecting Disulfide Bond Structures with intrachain A Cys(11)-A Cys(20) linkage, interchain A Cys(6)-B Cys(19) linkage, and interchain A Cys(7)-B Cys(7) linkage.

FIG. 6 is a schematic representation of the sequence of the abnormal Disulfide Bond Structures of abnormal human insulin NCI5 isoform including the A-and B-chains showing the connecting Disulfide Bond Structures with intrachain A Cys(7)-A Cys(20) linkage, interchain A Cys(11)-B Cys(19) linkage, and interchain A Cys(6)-B Cys(7) linkage.

FIG. 7 is a schematic representation of the sequence of the abnormal Disulfide Bond Structures of abnormal human insulin NCI6 isoform including the A-and B-chains showing the connecting Disulfide Bond Structures with interchain A Cys(7)-B Cys(19) linkage, interchain A Cys(20)-B Cys(7) linkage, and intrachain A Cys(6)-A Cys(11) linkage.

FIG. 8 is a workflow diagram that outlines the sequential steps in the control process for determining the level of Insulin Resistance and administering treatment. The workflow follows the sequence of actions required to analyze patient blood and determine the appropriate course of treatment based on assay results and concentration levels.

FIG. 9 is a schematic diagram that visually represents the components involved in the control process. This figure provides a structural view of the system, including assay components and pathways used to analyze the patient's blood sample and regulate treatment administration. While it does not include decision-making steps directly, the schematic shares labeling conventions with FIG. 8 for consistency and clarity. Both diagrams are aligned to show the same overall process, with FIG. 9 focused on the physical components and FIG. 8 illustrating the operational steps.

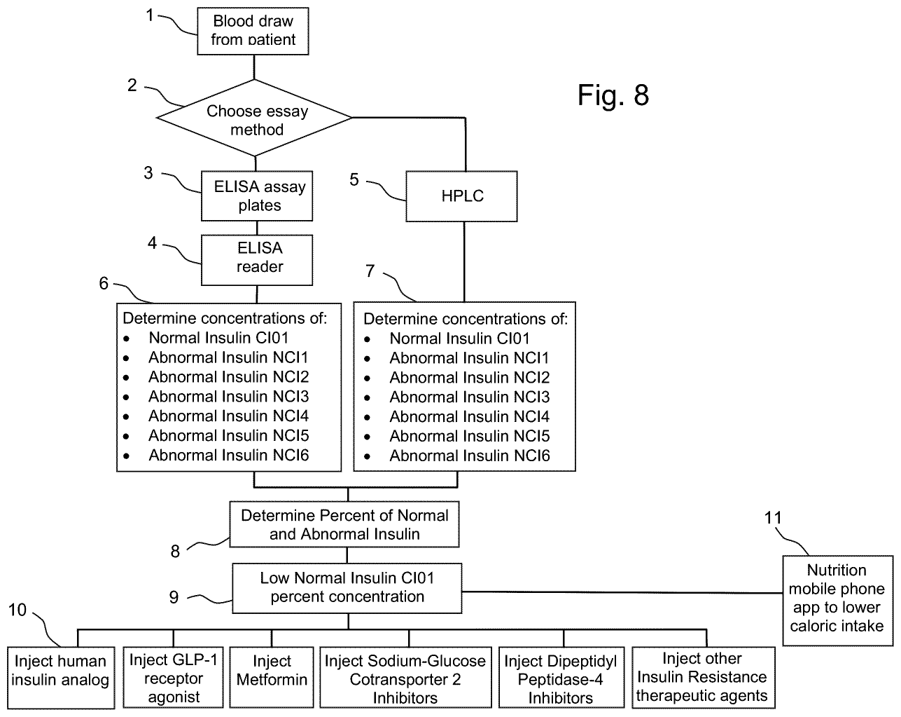

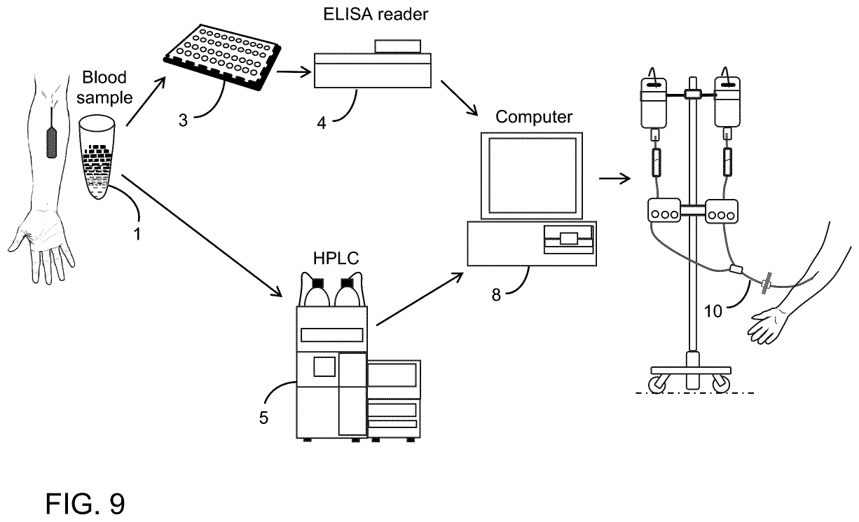

As illustrated in both FIG. 8 and FIG. 9, Step 1 initiates the process by a blood draw from patient. In FIG. 8, Step 2 involves making a decision based on availability, an operational aspect that is not represented in the schematic (FIG. 9). This step is included in the workflow diagram to show the complete process, even though it does not correspond to a particular symbol or component in the schematic. While FIG. 9 does not show the decision-making process directly, the pathways indicate potential processing of the patient blood either through ELISA assay components corresponding to Steps 3, 4, and 6 or alternatively through the HPLC assay as Step 5 and 7.

The human insulin isomers' concentration is determined in Steps 6 and 7 by respective assay type, and in Step 8 results information are fed into a computer system to determine percent of normal and abnormal insulin isoforms in patient's blood sample.

The information in Step 8 is used in Step 9 to make a determination whether the relative (percent) concentration of normal insulin is low and to regulate in Step 10 the level of exogenous insulin to augment the level of normal insulin detected by the methods for a specific patient, or regulate the amount of Metformin, sulfonylureas, thiazolidinediones (PPARy agonists; glitazones), GLP-1s, SGLT-2 inhibitors and DPP-4 inhibitors or other Insulin Resistance treatment to be administered to the patient.

In Step 11 the relative (percent) concentration of each type of insulin isoform is accessed by a mobile phone or tablet app that displays the levels of normal and abnormal insulin and when proportion (percent) of normal insulin is low makes recommendations to patient on how to decrease caloric intake by changing nutritional intake.

### DETAILED DESCRIPTION OF THE INVENTION

Various embodiments now will be described more fully hereinafter with reference to the accompanying drawings, which form a part hereof, and which show, by way of illustration, specific embodiments by which the invention may be practiced. The embodiments may, however, be embodied in many different forms and should not be construed as limited to the embodiments set forth herein; rather, these embodiments are provided so that this disclosure will be thorough and complete, and will fully convey the scope of the embodiments to those skilled in the art.

Throughout the specification and claims, the following terms take the meanings explicitly associated herein, unless the context clearly dictates otherwise. The term “herein” refers to the specification, claims, and drawings associated with the current application. The phrase “in an embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Described are methods and devices for measuring insulin resistance by utilizing monoclonal/polyclonal antibody to human insulin, and HPLC and kits adapted for use in such methods for modulation of treatment of insulin resistance.

An advantage of the present disclosure is the provision of a monoclonal and/or polyclonal antibody to normal and abnormal insulin and therefore, a new method of measuring Insulin Resistance, which overcomes the disadvantages of the current methods used. Antibodies will accurately identify, locate and bind to disulfide bonds that are normal and non-normal:
a) These abnormal disulfide bonds are the immunogens
b) The antibody reacts with human insulin
c) The antibody does not react with non-human insulin
d) The antibody does not react with human pro-insulin
e) The antibody does not react with human IGF-1 insulin precursor
f) The antibody does not react with insulin analog

The present disclosure further relates to an insulin assay reagent—antibody (monoclonal, polyclonal) and assay (detection and binding to one or more abnormal disulfide bonds), using the abnormal human insulin.

The following method relates to the content of insulin assay using human monoclonal and polyclonal antibodies.

Particularly, the present disclosure relates to a method of measuring normal and “abnormal” (as defined previously) human insulin that displays one or more abnormal disulfide bonds. We convey that this is a method of measuring IR, by determining the levels of “abnormal” and normal insulin.

The disulfide bond structure of human insulin is highly conserved through evolution and was considered a uniform and homogeneous structural feature. The structure of insulin is highly conserved in all vertebrates, and it is stabilized by three disulfide bonds. Detailed characterization of human insulin has revealed several new structural features in recombinant and natural human insulin, and non-classical disulfide bond structures have been described. Disulfide bonds are susceptible to chemical modifications, which can further generate structural variants, isomers, or new forms of insulin (an abnormal insulin). The effect of these disulfide bond variations on insulin structure and stability can result in different biochemical function.

Insulin contains two inter-chain disulfide bonds between the A and B chains (A7-B7 and A20-B19), and one intra-chain linkage in the A chain (A6-A11). Structural and biological studies of the insulin and the three disulfide bonds revealed that all three disulfide bonds are for the receptor binding activity of insulin. For example, deletion of the A20-B19 disulfide bond had the most substantial influence on the structure of human insulin, as indicated by the loss of ordered secondary structure, increased susceptibility to proteolysis, and markedly reduced compactness. When the A6-A11 disulfide was deleted, the result was the perturbation of the insulin structure. In addition, different refolding efficiencies between the three disulfide bonds, suggest that these bonds are formed sequentially in the order: A20-B19, A7-B7 and A6-A11, in the folding pathway of proinsulin.

The disulfide bond structures are referred to as the classical disulfide bond, formed between two Cysteine residues. In the case of human insulin, the A-chain and B-chain are connected by two disulfide bonds (A7-B7 and A20-B19). The lighter chain (A-chain) has one intra-chain disulfide bond (A6-A11).

The level of solvent exposure is different between intra-chain and inter-chain disulfide bonds. Cysteine residues that form inter-chain disulfide bonds are located in the hinge regions and therefore, inter-chain disulfide bonds are highly solvent exposed. The intra-chain disulfide bond is buried between the two layers of anti-parallel Beta-sheet structures, and are not solvent exposed, similar to IgG structure. The solvent exposure difference has implications, because exposed Cysteines are considered more reactive than non-exposed ones.

It has been shown by numerous studies the influence of each disulfide bond on the formation of other disulfide linkages during the folding process. The A20-B19 disulfide bond had the greatest effect on refolding, followed by the A7-B7 bond, and the intra-chain disulfide (A6-A11) had the least effect.

It has also been shown that all three disulfide bonds from the normal/normal human insulin may contribute to insulin activity by maintaining biologically active conformation.

Non-classical or abnormal disulfide bond structures were first identified in recombinant monoclonal antibodies (mAbs) and then confirmed in human IgG2 molecules. Molecular dynamic simulation study revealed that the sulfur atom of inter-chain disulfide bonds is highly mobile and can be in close proximity.

Similarly, some situation may happen in human insulin, resulting in the coexistence of multiple disulfide bond isoforms. As a result, the biological activity of the insulin is affected, resulting in non-binding of insulin-to-insulin receptor, hyperinsulinemia and IR, that will culminate with diabetes.

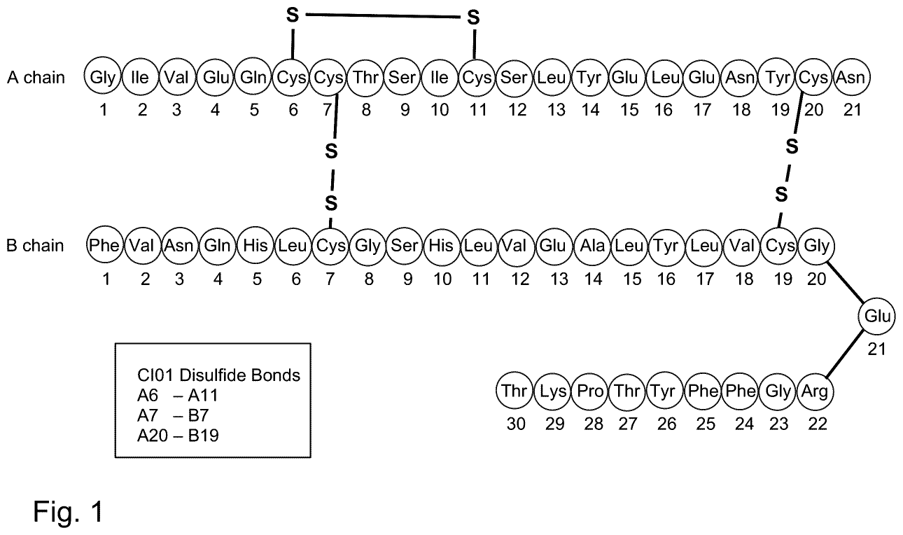

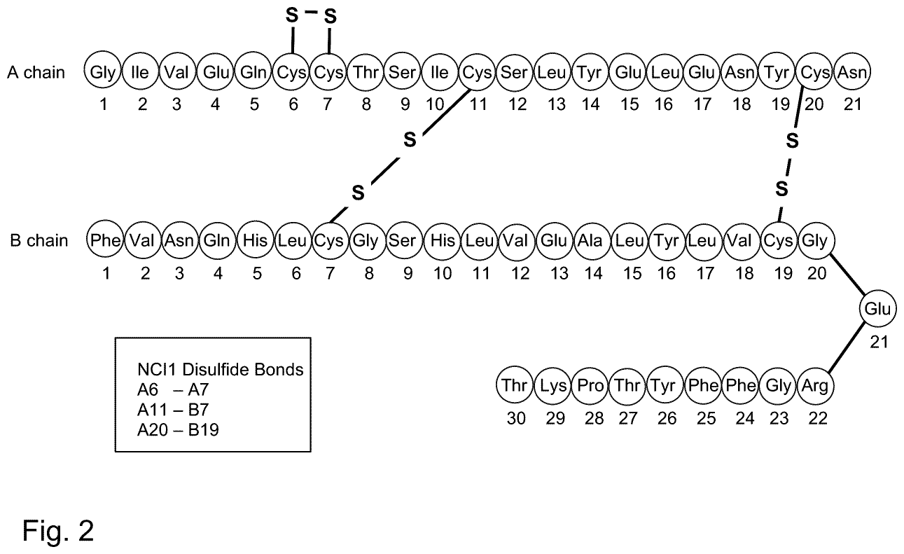

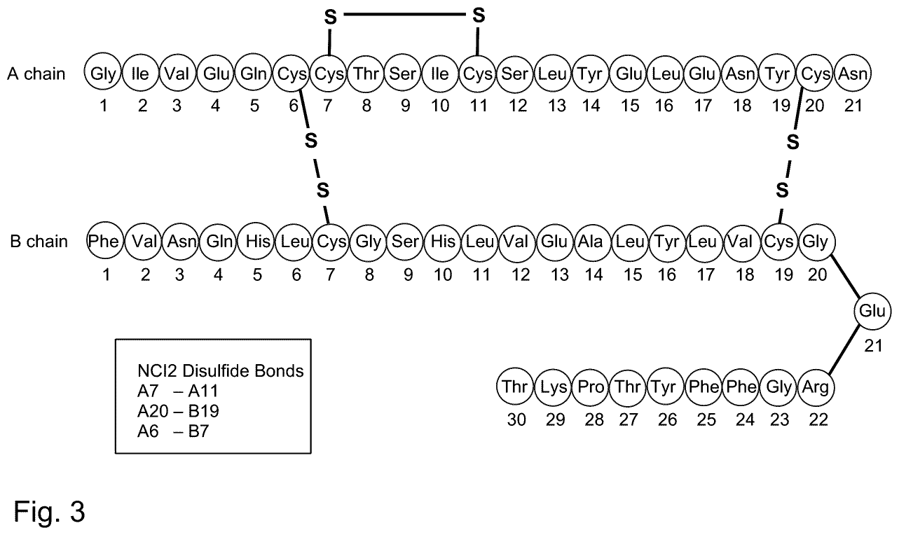

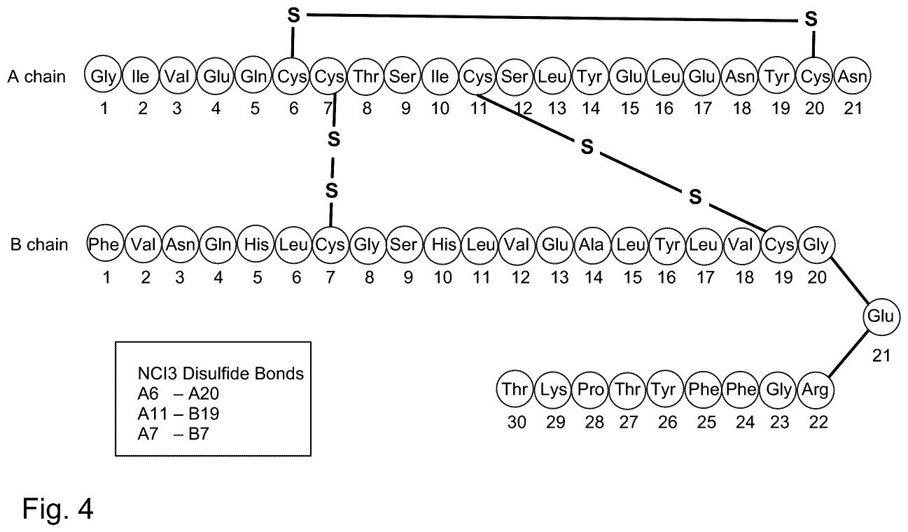

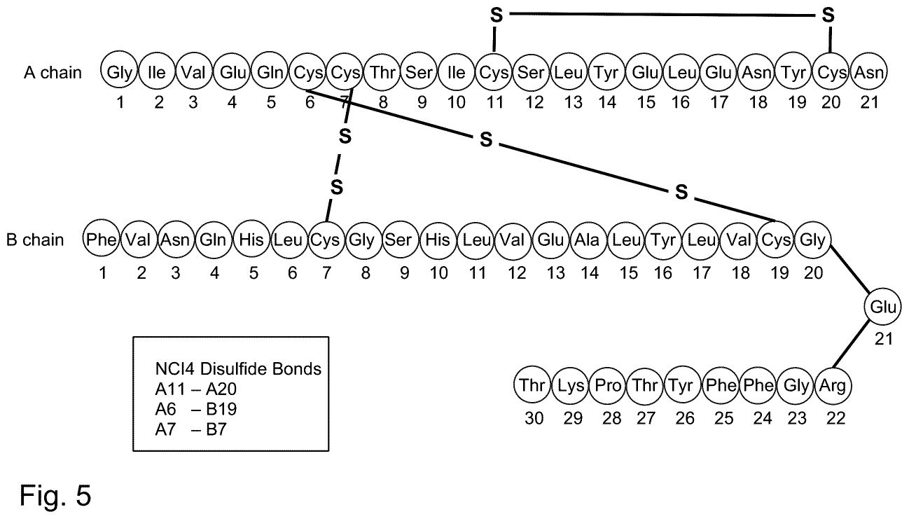

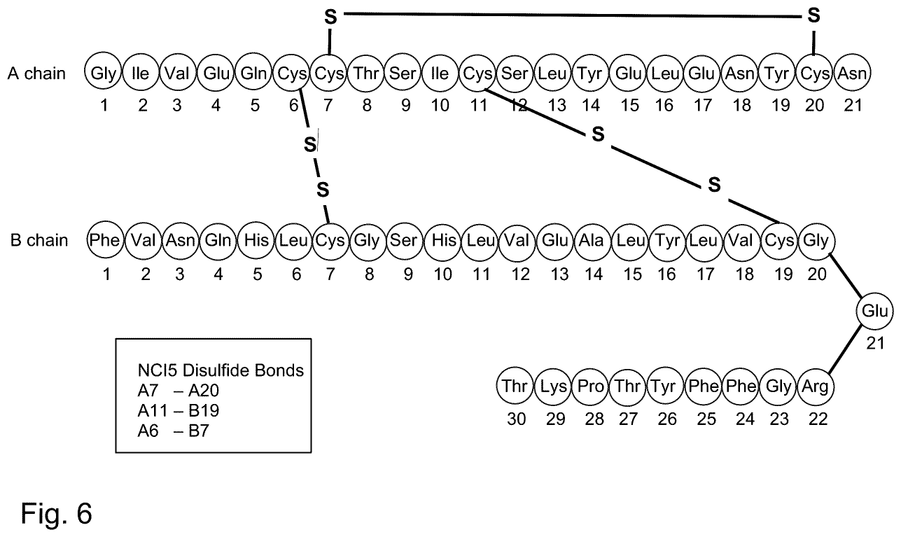

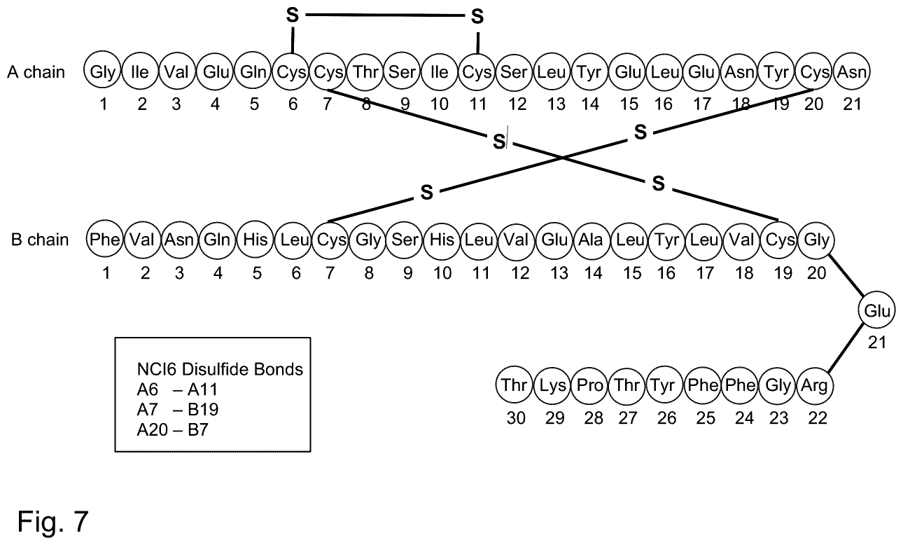

Correct disulfide bond formation is responsible for the biological activity of the protein. Mismatches or incomplete formation of disulfide bonds tend to decrease the protein's activity. FIG. 1 shows Disulfide Bond Structures in normal insulin. FIGS. 2-7 show abnormal Disulfide Bond Structures in normal insulin. FIG. 2 shows abnormal Disulfide Bond Structures in normal insulin NCI1. FIG. 3 shows abnormal Disulfide Bond structures in normal insulin NCI2. FIG. 4 shows abnormal Disulfide Bond Structures in normal insulin NCI3. FIG. 5 shows abnormal Disulfide Bond Structures in normal insulin NCI4. FIG. 6 shows abnormal Disulfide Bond Structures in normal insulin NCI5. FIG. 7 shows abnormal Disulfide Bond Structures in normal insulin NCI6.

Trisulfide bonding formation is a rare post-translational modification of proteins. The presence of trisulfide bonding was first reported for a recombinant monoclonal IgG2, where 1 or 2 of the 4 inter heavy chain disulfide bonds may exist as a trisulfide bond

A protein with an unaltered function is obtained via a correct/proper structure, or correct folding. The correct folding is associated with correct disulfide bond formation. In mammals, where protein folding is compartmentalized and complex, disulfide bond formation takes place in specialized organelles, such as the ER and mitochondria.

Under nutritional stress, the formation and secretion of insulin are more complex and vulnerable than in normal conditions. The formation and secretion of insulin in prediabetes stage, and in insulin resistance phase, can be impacted by many factors influencing insulin's functional properties including higher order structure modifications (e.g., misfolding, aggregation) and post-translational modifications (e.g. disulfide linkage scrambling, oxidation).

Incorrect pairing of disulfide bonds may affect the folding that can lead to a change in protein function, such as: antigen recognition, binding affinity, structure and stability.

Cells undergoing stress, such as pancreatic B-cells are prone to changes in post-translational modifications, alter-normal splicing, translational infidelity, and misfolding of proteins. All of these are the result of a disrupted normal equilibrium of the ER.

Commonly seen modifications in disulfide structures include disulfide bond scrambling, glutathionylation, cysteinylation, and oxidation. Trisulfide modifications of proteins, resulting from insertion of a sulfur atom into a disulfide bond (Cys—S—S—Cys=H2S+[O]→Cys—S—S—S—Cys+H₂O) have rarely been documented. The precise chemistry of the reaction has not been elucidated. The presence of a trisulfide in a protein was first reported for the minor disulfide loop of Escherichia Coli—derived recombinant human growth hormone (hGH).

It was found that after administration of H2S in Wistar rats, the blood glucose level increased and insulin level decreased in the first 5 hours post H2S. In ZDF model, H2S concentration was reported to be higher than in Zucker lean rats. Also, expressions of these enzymes responsible for H2S production—CSE and CBS—were high in streptozotocin-induced diabetic rats and elevated in patients with T2D. It was observed that chronic treatment of H2S for 1 month had no significant effects on insulin sensitivity and that the high H2S level in diabetes is due to overexpression of H2S.

The concentration of H2S in plasma or in tissue is regulated at the level of its generation and its consumption, and the levels are maintained within a certain range. Significant changes in the levels contribute to various diseases.

### Cause of Disulfide Bond Formation Considered

Disulfide bond formation inside the cell is spontaneous and that the amino acid sequence is sufficient to determine correct folding of the peptide or protein. Either a systematic network of enzymes (such as a disulfide bond generating enzyme, a disulfide donor enzyme), or a redox cofactor, that function inside the cell, dictates the formation and maintenance of disulfide bonds.

Commercial insulins refer to the insulin products manufactured and marketed by pharmaceutical companies and are recombinant proteins with correct folding. Misfolding is the prevalent form of the insulin of a type 2 diabetic patient and represents a principal difference to the insulin of a non-diabetic (healthy) individual, and also of a commercial insulin.

Diagnosis of insulin resistance based on the detection of different (15 isoforms) of “abnormal” disulfide bond pairs that may form in insulin, affecting the normal insulin structure.

The present disclosure relates to an antibody specifically reactive with “abnormal” forms of insulin and relates to an insulin assay using the human insulin and human monoclonal antibodies specifically reactive with the “abnormal insulin”.

“Abnormal insulin” can be any insulin that contains at least one of the 15 forms of “abnormal” disulfide bond pairs mentioned above. The method will measure human insulin in a sample utilizing a monoclonal or polyclonal antibody to human insulin that contains one or more “abnormal” disulfide bonds (“abnormal insulin”).

Non-classical disulfide bond structure was identified in IgG4 and later in IgG2 antibodies. Although, cysteine residues should be in the disulfide bonded states, free sulfhydryls have been detected in all subclasses of IgG antibodies.

In addition, disulfide bonds are susceptible to chemical modifications which can further generate structural variants, such as IgG antibodies with tri-sulfide bonds, or thioether linkages.

The intestines and B cells communicate extensively with each other (via hormonal and neuronal signals) to help match food intake with insulin output, and early B cell expansion, in order to promote life-long metabolic health.

Upon Signal Peptide (SP) removal, proinsulin begins to quickly form “normal” and isomeric disulfide bonds. Under conditions in which normal XP cleavage is interrupted, uncleaved pre-proinsulin exhibits delayed and abnormal oxidative folding causing the formation of disulfide-linked complexes. Impaired XP cleavage triggers defective oxidative folding within the ER, leaving exposed thiols on the pre-proinsulin that could create cellular problems by participating in inappropriate intermolecular thiol attack and impairing the oxidation of the natural (classical) insulin disulfide bonds. Formation of 3 disulfide bonds in pro-insulins may occur via transfer of reducing equivalents to ER oxidoreductases and from there to the cytosol to ER 01.

Described are embodiments of a method of measuring Insulin Resistance (IR). In embodiments, the formation of insulin isoforms (antigens) is identified as an underlying cause of IR. In embodiments, the formation of disulfide bonds within the insulin are identified as an underlying cause of IR. In embodiments, described is the creation of an antibody (Ab) that will combine with the multivalent antigen (insulin isoforms).

Accordingly, described are embodiments of a system and method for measuring Insulin Resistance by identifying and quantifying the formation of insulin isoforms.

### First Embodiment

FIG. 1 shows Disulfide Bond Structures in normal insulin. In a first embodiment, described is an anti-human insulin antibody having the following properties:
a) the antibody reacts with normal human insulin CI01 isoform in FIG. 1.
   The anti-human insulin antibody can react with normal human insulin CI01 isoform in FIG. 1 and can further have one or more of the following properties:
b) the antibody does not react with abnormal human insulin NCI1 isoform,
c) the antibody does not react with abnormal human insulin NCI2 isoform,
d) the antibody does not react with abnormal human insulin NCI3 isoform,
e) the antibody does not react with abnormal human insulin NCI4 isoform,
f) the antibody does not react with abnormal human insulin NCI5 isoform,
g) the antibody does not react with abnormal human insulin NCI6 isoform,
h) the antibody does not react with human pre proinsulin,
i) the antibody does not react with human proinsulin,
j) the antibody does not react with a human insulin analog, and
k) the antibody does not react with non-human insulins.

The anti-human insulin antibody that reacts with normal human insulin CI01 isoform in FIG. 1, can be a monoclonal antibody. The anti-human insulin antibody can be produced by a hybridoma.

### Second Embodiment

FIG. 2 shows abnormal Disulfide Bond Structures in normal insulin NCI1. In a second embodiment, the anti-human insulin antibody can have the following properties:
a) the antibody reacts with abnormal human NCI1 isoform in FIG. 2.
   The anti-human insulin antibody can react with abnormal human NCI1 isoform in FIG. 2 and can further have one or more of the following properties: the antibody does not react with insulin normal human insulin CI01 isoform, and
b) the antibody does not react with abnormal human insulin NCI2 isoform,
c) the antibody does not react with abnormal human insulin NCI3 isoform,
d) the antibody does not react with abnormal human insulin NCI4 isoform,
e) the antibody does not react with abnormal human insulin NCI5 isoform,
f) the antibody does not react with abnormal human insulin NCI6 isoform,
g) the antibody does not react with human pre proinsulin,
h) the antibody does not react with human proinsulin,
i) the antibody does not react with a human insulin analog,
j) the antibody does not react with non-human insulins.

In the second embodiment, the anti-human insulin antibody having the properties (a) and one or more of the properties selected from (b)-(k) can be a monoclonal antibody. The anti-human insulin antibody of the second embodiment can be produced by a hybridoma.

### Third Embodiment

FIG. 3 shows abnormal Disulfide Bond structures in normal insulin NCI2. In a third embodiment, described is an anti-human insulin antibody having the following property: a) the antibody reacts with abnormal human NCI2 isoform in FIG. 3. In the third embodiment, the anti-human insulin antibody reacts with abnormal human NCI2 isoform in FIG. 3 and further has one or more of the following properties:
b) the antibody does not react with insulin normal human insulin CI01 isoform,
c) the antibody does not react with abnormal human insulin NCI1 isoform,
d) the antibody does not react with abnormal human insulin NCI3 isoform,
e) the antibody does not react with abnormal human insulin NCI4 isoform,
f) the antibody does not react with abnormal human insulin NCI5 isoform,
g) the antibody does not react with abnormal human insulin NCI6 isoform,
h) the antibody does not react with human pre-proinsulin,
i) the antibody does not react with human proinsulin,
j) the antibody does not react with a human insulin analog,
k) the antibody does not react with non-human insulins

The anti-human insulin antibody of the third embodiment can be a monoclonal antibody. The anti-human insulin antibody of the third embodiment can be produced by a hybridoma.

### Fourth Embodiment

FIG. 4 shows abnormal Disulfide Bond Structures in normal insulin NCI3. In a fourth embodiment, an anti-human insulin antibody (a) reacts with an abnormal human NCI3 isoform as shown in FIG. 4. The anti-human insulin antibody that reacts with an abnormal human NCI3 isoform as shown in FIG. 4 can further have one or more of the following properties:
b) the antibody does not react with insulin normal human insulin CI01 isoform,
c) the antibody does not react with abnormal human insulin NCI1 isoform,
d) the antibody does not react with abnormal human insulin NCI2 isoform,
e) the antibody does not react with abnormal human insulin NCI4 isoform,
f) the antibody does not react with abnormal human insulin NCI5 isoform,
g) the antibody does not react with abnormal human insulin NCI6 isoform,
h) the antibody does not react with human pre proinsulin,
i) the antibody does not react with human proinsulin,
j) the antibody does not react with a human insulin analog,
k) the antibody does not react with non-human insulins.

The anti-human insulin antibody of the fourth embodiment can be a monoclonal antibody. The anti-human insulin antibody of the fourth embodiment can be produced by a hybridoma.

### Fifth Embodiment

FIG. 5 shows abnormal Disulfide Bond Structures in normal insulin NCI4. In a fifth embodiment anti-human insulin antibody comprises (a) an antibody that reacts with abnormal human NCI4 isoform in FIG. 5. In the fifth embodiment, the anti-human insulin that reacts with abnormal human NCI4 isoform in FIG. 5 further has one or more of the following properties:
b) the antibody does not react with insulin normal human insulin CI01 isoform,
c) the antibody does not react with abnormal human insulin NCI1 isoform,
d) the antibody does not react with abnormal human insulin NCI2 isoform,
e) the antibody does not react with abnormal human insulin NCI3 isoform,
f) the antibody does not react with abnormal human insulin NCI5 isoform,
g) the antibody does not react with abnormal human insulin NCI6 isoform,
h) the antibody does not react with human pre proinsulin,
i) the antibody does not react with human proinsulin,
j) the antibody does not react with a human insulin analog,
k) the antibody does not react with non-human insulins.

The anti-human insulin antibody of the fifth embodiment can be a monoclonal antibody. The anti-human insulin antibody of the fifth embodiment can be produced by a hybridoma.

### Sixth Embodiment

FIG. 6 shows abnormal Disulfide Bond Structures in normal insulin NCI5. In a sixth embodiment, an anti-human insulin antibody comprises an antibody having the following property: a) the antibody reacts with abnormal human NCI5 isoform in FIG. 6. In the sixth embodiment, the anti-human insulin antibody that reacts with abnormal human NCI5 isoform in FIG. 6 further has one or more of the following properties:
b) the antibody does not react with insulin normal human insulin CI01 isoform,
c) the antibody does not react with abnormal human insulin NCI1 isoform,
d) the antibody does not react with abnormal human insulin NCI2 isoform,
e) the antibody does not react with abnormal human insulin NCI3 isoform,
f) the antibody does not react with abnormal human insulin NCI4 isoform,
g) the antibody does not react with abnormal human insulin NCI6 isoform,
h) the antibody does not react with human pre proinsulin,
i) the antibody does not react with human proinsulin,
j) the antibody does not react with a human insulin analog,
k) the antibody does not react with non-human insulin.
   The anti-human insulin antibody of the sixth embodiment can be a monoclonal antibody.
   The anti-human insulin antibody of the sixth embodiment can be produced by a hybridoma.

### Seventh Embodiment

FIG. 7 shows abnormal Disulfide Bond Structures in normal insulin NCI6. In a seventh embodiment, an anti-human insulin antibody comprises an anti-human insulin antibody having the following property:
a) the antibody reacts with abnormal human NCI6 isoform in FIG. 7.
   In the seventh embodiment, the anti-human insulin antibody that reacts with abnormal human NCI6 isoform in FIG. 7 further has one or more of the following properties:
b) the antibody does not react with insulin normal human insulin CI01 isoform,
c) the antibody does not react with abnormal human insulin NCI1 isoform,
d) the antibody does not react with abnormal human insulin NCI2 isoform,
e) the antibody does not react with abnormal human insulin NCI3 isoform,
f) the antibody does not react with abnormal human insulin NCI4 isoform,
g) the antibody does not react with abnormal human insulin NCI5 isoform,
h) the antibody does not react with human pre proinsulin,
i) the antibody does not react with human proinsulin,
j) the antibody does not react with a human insulin analog,
k) the antibody does not react with non-human insulins.

The anti-human insulin antibody of the seventh embodiment can be a monoclonal antibody. The anti-human insulin antibody of the seventh embodiment can be produced by a hybridoma.

### Eighth Embodiment

In an embodiment, described is a human insulin assay comprising a step of bringing the antibodies into contact with a subject's biological sample to detect a complex of the antibody and human insulin formed by the contact, wherein the antibodies include one or more antibodies of the first, second, third, fourth, fifth, sixth and seventh embodiments which are labeled with a detectable labeling material. Specifically, a human insulin assay reagent using the following antibodies:
1) an anti-human insulin antibody of the first, second, third, fourth, fifth, sixth and seventh embodiments, and
2) an antibody A having a property of specifically recognizing the antibody of 1).

The antibodies of 1) and 2) of the human insulin assay reagent can be monoclonal antibodies. In another embodiment, the antibody of 1) can be a monoclonal antibody, and the antibody of 2) can be a polyclonal antibody. The antibody of 1) and/or the antibody of 2) can be immobilized to a solid phase. The solid phase can be latex, and wherein insulin can be assayed by a latex immuno-agglutination assay. When the antibody of 1) is immobilized to a solid phase, the antibody of 2) can be labeled with a labeling material, and the insulin can be assayed by ELISA or immunochromatography.

In an embodiment, the human insulin assay can be employed to obtain a relative normal human insulin concentration value using the steps of: (1) obtaining a total concentration of human insulin; (2) obtaining specific concentration of normal human insulin concentration and/or of abnormal human insulin isomers with the insulin assay; and (3) obtaining a relative normal human insulin concentration by subtracting the concentration obtained at step (2) from the concentration obtained at step (1) and dividing by the concentration obtained at step (1).

Embodiments as described herein can be employed in a diagnostic method and systems and apparatuses therefor. FIGS. 8 and 9 depict a process for determining the level of Insulin Resistance in a patient and administering Insulin Resistance treatment based on assay results. The diagrams provide two perspectives of the same process: FIG. 8 outlines the sequential workflow steps, while FIG. 9 focuses on the system's physical components. Both figures share the same labeling conventions to ensure a unified representation of the process.

As illustrated in both FIG. 8 and FIG. 9, the process begins with Step 1, where a blood sample is drawn from the patient. In the workflow diagram (FIG. 8), Step 2 includes a decision-making step regarding the availability of specific assays, an operational detail that is not represented in the schematic (FIG. 9). This decision allows flexibility in processing the blood sample through different assay types depending on availability.

After Step 2, the blood sample may be processed through one or more assay pathways. For example, it could proceed through the ELISA assay, shown in Steps 3, 4,and 6, or through the HPLC assay, represented by Steps 5 and 7. These assays determine the concentration of human insulin isomers' concentration in the patient's blood sample. The workflow (FIG. 8) includes the decision-making process based on assay availability, while the schematic (FIG. 9) illustrates the components involved in these assays.

In both assays, the concentration of human insulin isomers' is determined in Steps 6 (ELISA assay) and Step 7 (HPLC assay). Once the concentration data is obtained, the results are fed into a computer system in Step 8. The system uses this information to calculate the percentage of normal and abnormal insulin isoforms in the patient's blood.

The process continues in Step 9, where a determination is made based on whether the relative concentration of normal insulin is low. This step informs Step 10, where the level of exogenous insulin administered to the patient is regulated to augment the proportion of normal insulin in the patient's blood. Alternatively, the concentration data can also be used to regulate the amount of Metformin, sulfonylureas, thiazolidinediones (PPARy agonists; glitazones), GLP-1s, SGLT-2 inhibitors and DPP-4 inhibitors or other Insulin Resistance treatments to be administered to the patient while also allowing medical team oversight of treatment based on patient specific factors.

In Step 11, the relative concentrations of both normal and abnormal insulin are accessed via a mobile phone or tablet app, which displays the data. When the proportion of normal insulin is low, the app can make recommendations to the patient regarding adjustments to their nutritional intake or other lifestyle changes to improve insulin levels.

Together, FIG. 8 and FIG. 9 provide a comprehensive view of the disease control and treatment process, with FIG. 8 focusing on the operational workflow and FIG. 9 depicting the system's structural components and pathways for analyzing patient blood samples and administering treatment.

Table 1, as an example, shows the results for normal patients with no Insulin Resistance and a high percentage of normal insulin—the human insulin isomers' concentrations as determined in Steps 6 and 7 and the percent of each insulin isoform concentration calculated in Step 8. For patients with high percentage of normal insulin isomer, such as patients A1-A7, with normal insulin isoform between 40%-100% there will not be any Insulin Resistance treatment such as Metformin (Treatment A=0 mg/day), Glucagon-like peptide-1 receptor agonist (Treatment B=0 μg/day), or other Insulin Resistance treatments, as referred in Table 1 (continued).

TABLE 1

| | | | | | | | | Normal |
|---|---|---|---|---|---|---|---|---|
| | | Abnormal isoforms | | | | | | isoform |
| | | Abnormal | Abnormal | Abnormal | Abnormal | Abnormal | Abnormal | Normal |
| | | human | human | human | human | human | human | human |
| | Total | insulin | insulin | insulin | insulin | insulin | insulin | insulin |
| | human | isoform | isoform | isoform | isoform | isoform | isoform | isoform |
| | insulin | NCI1 | NCI02 | NCI03 | NCI04 | NCI05 | NCI06 | CI01 |
| Patient | (mU/L) | (mU/L) | (mU/L) | (mU/L) | (mU/L) | (mU/L) | (mU/L) | (mU/L) |
| A1 | 3.02 | 0.14 | 0.57 | 0.9 | 0.02 | 0.98 | 0.42 | 9.52 |
| A2 | 2.71 | 0.48 | 0.2 | 0.9 | 0.49 | 0.06 | 0.59 | 13.67 |
| A3 | 1.97 | 0.27 | 0.25 | 0.35 | 0.4 | 0.18 | 0.52 | 9.69 |
| A4 | 2.17 | 0.15 | 0.8 | 0.91 | 0.08 | 0.22 | 0.01 | 16.82 |
| A5 | 2.73 | 0.21 | 0.62 | 0.69 | 0.16 | 0.38 | 0.67 | 11.04 |
| A6 | 3.08 | 0.03 | 0.63 | 0.71 | 0.43 | 0.66 | 0.61 | 14.21 |
| A7 | 2.75 | 0.14 | 0.28 | 0.25 | 0.35 | 0.85 | 0.88 | 14.09 |

| | | | | | | | Normal | | Treatment |
|---|---|---|---|---|---|---|---|---|---|
| | Abnormal isoforms (% of total insulin) | | | | | | isoform | | B |
| | Abnormal | Abnormal | Abnormal | Abnormal | Abnormal | Abnormal | Normal | | Glucagon- |
| | human | human | human | human | human | human | human | Treatment | like peptide-1 |
| | insulin | insulin | insulin | insulin | insulin | insulin | insulin | A | receptor |
| | isoform | isoform | isoform | isoform | isoform | isoform | isoform | Metformin | agonist |
| Patient | NCI1 | NCI02 | NCI03 | NCI04 | NCI05 | NCI06 | CI01 | (mg/day | (μg/day) |
| A1 | 1% | 6% | 9% | 0% | 10% | 4% | 68% | 0 | 0 |
| A2 | 4% | 1% | 7% | 4% | 0% | 4% | 80% | 0 | 0 |
| A3 | 3% | 3% | 4% | 4% | 2% | 5% | 80% | 0 | 0 |
| A4 | 1% | 5% | 5% | 0% | 1% | 0% | 87% | 0 | 0 |
| A5 | 2% | 6% | 6% | 1% | 3% | 6% | 75% | 0 | 0 |
| A6 | 0% | 4% | 5% | 3% | 5% | 4% | 78% | 0 | 0 |
| A7 | 1% | 2% | 2% | 2% | 6% | 6% | 80% | 0 | 0 |

Table 2 shows the results for patients with Insulin Resistance and a low percentage of normal insulin—the human insulin isomers' concentrations as determined in Steps 6 and 7 and the percent of each insulin isoform concentration calculated in Step 8. For patients with low percentage of normal insulin isomer, such as patients B1-B7, with normal insulin isoform <40% there will be Insulin Resistance treatment such as Metformin (Treatment A), Glucagon-like peptide-1 receptor agonist (Treatment B), or other Insulin Resistance treatments commensurate with the percent level of normal insulin isomer, as referred in Table 2 (continued).

TABLE 2

| | | | | | | | | Normal |
|---|---|---|---|---|---|---|---|---|
| | | Abnormal isoforms | | | | | | isoform |
| | | Abnormal | Abnormal | Abnormal | Abnormal | Abnormal | Abnormal | Normal |
| | | human | human | human | human | human | human | human |
| | Total | insulin | insulin | insulin | insulin | insulin | insulin | insulin |
| | human | isoform | isoform | isoform | isoform | isoform | isoform | isoform |
| | insulin | NCI1 | NCI02 | NCI03 | NCI04 | NCI05 | NCI06 | CI01 |
| Patient | (mU/L) | (mU/L) | (mU/L) | (mU/L) | (mU/L) | (mU/L) | (mU/L) | (mU/L) |
| B1 | 144.98 | 19.40 | 7.15 | 0.88 | 30.20 | 16.88 | 28.44 | 42.04 |
| B2 | 47.06 | 6.26 | 6.34 | 3.56 | 6.95 | 4.18 | 6.60 | 13.18 |
| B3 | 160.17 | 17.51 | 33.32 | 34.86 | 2.63 | 16.61 | 5.59 | 49.65 |
| B4 | 90.76 | 5.05 | 17.39 | 12.70 | 5.74 | 0.71 | 15.59 | 33.58 |
| B5 | 172.40 | 21.38 | 9.96 | 16.05 | 30.95 | 34.87 | 19.53 | 39.65 |
| B6 | 44.39 | 7.05 | 10.06 | 6.82 | 1.40 | 0.53 | 0.78 | 17.76 |
| B7 | 44.19 | 8.10 | 6.09 | 0.21 | 5.95 | 2.16 | 5.35 | 16.35 |

| | | | | | | | Normal | | |
|---|---|---|---|---|---|---|---|---|---|
| | | | | | | | isoform | | |
| | | | | | | | (% of | | Treatment |
| | | | | | | | total | | B |
| | Abnormal isoforms (% of total insulin) | | | | | | insulin) | | Glucagon- |
| | Abnormal | Abnormal | Abnormal | Abnormal | Abnormal | Abnormal | Normal | | like |
| | human | human | human | human | human | human | human | Treatment | peptide-1 |
| | insulin | insulin | insulin | insulin | insulin | insulin | insulin | A | receptor |
| | isoform | isoform | isoform | isoform | isoform | isoform | isoform | Metformin | agonist |
| Patient | NCI1 | NCI02 | NCI03 | NCI04 | NCI05 | NCI06 | CI01 | (mg/day | (μg/day) |
| B1 | 13% | 5% | 1% | 21% | 12% | 20% | 29% | 2,167 | 22 |
| B2 | 13% | 13% | 8% | 15% | 9% | 14% | 28% | 2,244 | 23 |
| B3 | 11% | 21% | 22% | 2% | 10% | 3% | 31% | 2,027 | 21 |
| B4 | 6% | 19% | 14% | 6% | 1% | 17% | 37% | 1,698 | 17 |
| B5 | 12% | 6% | 9% | 18% | 20% | 11% | 23% | 2,732 | 28 |
| B6 | 16% | 12% | 15% | 3% | 1% | 2% | 40% | 1,571 | 16 |
| B7 | 18% | 14% | 0% | 13% | 5% | 12% | 37% | 1,698 | 17 |

The foregoing examples and embodiments should not be construed as limiting and/or exhaustive, but rather, illustrative to show implementations of various embodiments of the disclosure.

It will be understood that some steps of the flowchart illustrations, and combinations of steps in the flowchart illustrations, can be implemented by computer program instructions. These program instructions may be provided to a processor to produce a machine, such that the instructions, which execute on the processor, create means for implementing the actions specified in the flowchart block or blocks. The computer program instructions may be executed by a processor to cause a series of operational steps to be performed by the processor to produce a computer-implemented process such that the instructions, which execute on the processor to provide steps for implementing the actions specified in the flowchart steps. The computer program instructions may also cause at least some of the operational steps of the flowchart to be performed in parallel. Moreover, some of the steps may also be performed across more than one processor, such as might arise in a multi-processor computer system or even a group of multiple computer systems. In addition, one or more steps or combinations of steps in the flowchart illustrations may also be performed concurrently with other steps or combinations of steps, or even in a different sequence than illustrated without departing from the scope or spirit of the invention.

Accordingly, steps of the flowchart illustrations support combinations of means for performing the specified actions, combinations of steps for performing the specified actions and program instruction means for performing the specified actions. It will also be understood that each block of the flowchart illustrations, and combinations of blocks in the flowchart illustrations, can be implemented by special purpose hardware-based systems, which perform the specified actions or steps, or combinations of special purpose hardware and computer instructions.

1. A method and system of monitoring the progression or regression of insulin resistance in a subject, the method comprising: analyzing a biological sample from a subject to determine levels of insulin isomers in the sample; and comparing the levels of insulin isomers in the sample to insulin resistance progression and/or insulin resistance-regression reference levels of insulin isomers in order to monitor the progression or regression of insulin resistance in a subject.

2. The method of claim 1, wherein the method further comprises analyzing the biological sample to determine the level(s) of an anti-human insulin monoclonal antibody produced by a hybridoma comprising the following properties of reacting with normal human CI01 isoform, and further having one or more of the following properties
of not reacting with abnormal human insulin NCI1 isoform,
of not reacting with abnormal human insulin NCI2 isoform,
of not reacting with abnormal human insulin NCI3 isoform,
of not reacting with abnormal human insulin NCI4 isoform,
of not reacting with abnormal human insulin NCI5 isoform,
of not reacting with abnormal human insulin NCI6 isoform,
of not reacting with human pre proinsulin,
of not reacting with human proinsulin,
of not reacting with a human insulin analog,
of not reacting with non-human insulins.

3. The method of claim 1, wherein the method further comprises analyzing the biological sample to determine the level(s) of one or more additional biomarkers selected from the group consisting of an anti-human insulin monoclonal antibody produced by a hybridoma comprising the property of reacting with abnormal human NCI1 isoform, and further having one or more of the following properties
of not reacting with insulin normal human insulin CI01 isoform,
of not reacting with abnormal human insulin NCI2 isoform,
of not reacting with abnormal human insulin NCI3 isoform,
of not reacting with abnormal human insulin NCI4 isoform,
of not reacting with abnormal human insulin NCI5 isoform,
of not reacting with abnormal human insulin NCI6 isoform,
of not reacting with human pre proinsulin,
of not reacting with human proinsulin,
of not reacting with a human insulin analog,
of not reacting with non-human insulins.

4. The method of claim 1, wherein the method further comprises analyzing the biological sample to determine the level(s) of an anti-human insulin monoclonal antibody produced by a hybridoma comprising the property of reacting with abnormal human NCI2 isoform, and further having one or more of the following properties
of not reacting with insulin normal human insulin CI01 isoform,
of not reacting with abnormal human insulin NCI1 isoform,
of not reacting with abnormal human insulin NCI3 isoform,
of not reacting with abnormal human insulin NCI4 isoform,
of not reacting with abnormal human insulin NCI5 isoform,
of not reacting with abnormal human insulin NCI6 isoform,
of not reacting with human pre proinsulin,
of not reacting with human proinsulin,
of not reacting with a human insulin analog,
of not reacting with non-human insulins.

5. The method of claim 1, wherein the method further comprises analyzing the biological sample to determine the level(s) of an anti-human insulin monoclonal antibody produced by a hybridoma comprising the property of reacting with abnormal human NCI3 isoform, and further having one or more of the following properties
of not reacting with insulin normal human insulin CI01 isoform,
of not reacting with abnormal human insulin NCI1 isoform,
of not reacting with abnormal human insulin NCI2 isoform,
of not reacting with abnormal human insulin NCI4 isoform,
of not reacting with abnormal human insulin NCI5 isoform,
of not reacting with abnormal human insulin NCI6 isoform,
of not reacting with human pre proinsulin,
of not reacting with human proinsulin,
of not reacting with a human insulin analog,
of not reacting with non-human insulins.

6. The method of claim 1, wherein the method further comprises analyzing the biological sample to determine the level(s) of an anti-human insulin monoclonal antibody produced by a hybridoma comprising the property of reacting with abnormal human NCI4 isoform, and further having one or more of the following properties
of not reacting with insulin normal human insulin CI01 isoform,
of not reacting with abnormal human insulin NCI1 isoform,
of not reacting with abnormal human insulin NCI2 isoform,
of not reacting with abnormal human insulin NCI3 isoform,
of not reacting with abnormal human insulin NCI5 isoform,
of not reacting with abnormal human insulin NCI6 isoform,
of not reacting with human pre proinsulin,
of not reacting with human proinsulin,
of not reacting with a human insulin analog,
of not reacting with non-human insulins.

7. The method of claim 1, wherein the method further comprises analyzing the biological sample to determine the level(s) of an anti-human insulin monoclonal antibody produced by a hybridoma comprising the properties of reacting with abnormal human NCI5 isoform, and further having one or more of the following properties
of not reacting with insulin normal human insulin CI01 isoform,
of not reacting with abnormal human insulin NCI1 isoform,
of not reacting with abnormal human insulin NCI2 isoform,
of not reacting with abnormal human insulin NCI3 isoform,
of not reacting with abnormal human insulin NCI4 isoform,
of not reacting with abnormal human insulin NCI6 isoform,
of not reacting with human pre proinsulin,
of not reacting with human proinsulin,
of not reacting with a human insulin analog,
of not reacting with non-human insulins.

8. The method of claim 1, wherein the method further comprises analyzing the biological sample to determine the level(s) of an anti-human insulin monoclonal antibody produced by a hybridoma comprising the following properties of reacting with abnormal human NC16 isoform, and further having one or more of the following properties
of not reacting with insulin normal human insulin CI01 isoform,
of not reacting with abnormal human insulin NCI1 isoform,
of not reacting with abnormal human insulin NCI2 isoform,
of not reacting with abnormal human insulin NCI3 isoform,
of not reacting with abnormal human insulin NCI4 isoform,
of not reacting with abnormal human insulin NCI5 isoform,
of not reacting with human pre proinsulin,
of not reacting with human proinsulin,
of not reacting with a human insulin analog,
of not reacting with non-human insulins.

9. A human insulin assay method comprising a step of bringing the antibodies of claim 2, 3, 4, 5, 6, 7, or 8 into contact with a biological sample to detect a complex of one or a plurality of the antibodies and human insulin.

10. The human insulin assay of claim 9, wherein the antibody complex is labeled with a detectable labeling material.

11. A human insulin assay method, comprising the following two antibodies:
the anti-human insulin antibody of claim 2, 3, 4, 5, 6, 7, or 8 and
an antibody A having a property of reacting at least with human insulin.

12. A human insulin assay method, comprising using the anti-human insulin monoclonal antibodies of claim 2, 3, 4, 5, 6, 7, or 8, and a polyclonal or monoclonal antibody B having a property of selectively binding to the antibodies.

13. The human insulin assay of claim 12, wherein the antibodies are immobilized onto a solid phase, wherein the solid phase comprises latex or other suitable materials, and wherein insulin is assayed using a latex immunoagglutination, ELISA, immunochromatography or other suitable assays.

14. A method for analyzing Insulin Resistance and modulating of Type 2 Diabetes onset treatment, the method comprising: analyzing a biological sample from a subject processed through assays in claim 13, High Performance Liquid Chromatography (HPLC) assay or other Liquid Chromatography assays to determine levels of types of human insulin isomers, wherein this information is fed into a computer system to compare with one or more normal baseline levels of types of human insulin isomers.

15. The method of claim 14, wherein the method further comprises analyzing the subject and a normal baseline biological sample using a mathematical model comprising levels of types of human insulin isomers to assess and modulate the effectiveness of the treatment for treating onset of Type 2 Diabetes.

16. The method of claim 14, wherein the treatment comprises administering a therapeutic agent to the subject by regulating the amount of exogenous insulin, metformin, sulfonylureas, thiazolidinediones (PPARy agonists; glitazones), GLP-1s, SGLT-2 inhibitors and DPP-4 inhibitors or other Type 2 Diabetes therapeutic agents administered to the subject.

17. The method of claim 14, wherein the biological sample is a blood/plasma sample.

18. The method of claim 14, wherein the treatment comprises a lifestyle modification of the subject.
